(19) United States
(12) Patent Application Publication
(10) Pub. No.: US 2019/0134460 A1
(43) Pub. Date: May 9, 2019

(54) RESPIRATORY THERAPY DEVICE AND SYSTEM WITH INTEGRATED GAMING CAPABILITIES AND METHOD OF USING THE SAME

(71) Applicants: Dwight Cheu, Albany, NY (US); Michael DiCesare, Albany, NY (US)

(72) Inventors: Dwight Cheu, Albany, NY (US); Michael DiCesare, Albany, NY (US)

(21) Appl. No.: 16/182,864

(22) Filed: Nov. 7, 2018

Related U.S. Application Data
(60) Provisional application No. 62/582,534, filed on Nov. 7, 2017.

(51) Int. Cl. A63B 23/18; A63B 24/00; A63F 13/212; A63F 13/23; A63F 13/40

(52) U.S. Cl. CPC A63B 23/185; A63B 24/0062; A63B 24/0075; A61M 15/009; A63F 13/23; A63F 13/40; A63F 13/212

(57) ABSTRACT

A processor-based respiratory device for respiratory therapy that combines gaming and real-time feedback to guide a user through proper respiratory techniques is provided. The device has a chamber being positioned inside of the housing between the inlet and the outlet of the housing, the chamber being configured to allow air to flow from a user to a form factor when the user breathes into the chamber; at least a sensor positioned within the chamber and electronically coupled to the processor, a connection member configured to form a seal with an opening of the form factor, and the processor comprises a communications interface coupled to a network, the communications interface being configured to output a signal to a graphical user interface based on the airflow in the chamber.

### CROSS-REFERENCE TO RELATED APPLICATIONS

This application claims the benefit of U.S. Provisional Application No. 62/582,534 entitled Respiratory Therapy Device with Integrated Gaming Capabilities, filed on Nov. 7, 2017.

### FIELD OF THE INVENTION

The present invention relates generally to a device, system and method of respiratory therapy. More particularly, the present invention relates to certain new and useful advances in respiratory therapy that uses a substrate device to combine gaming with respiratory therapy to provide an engaging experience thereby ensuring that a user receives the maximum health benefit possible from a particular respiratory therapy routine.

### BACKGROUND OF THE INVENTION

Many people with chronic lung diseases such as asthma, cystic fibrosis, emphysema, pulmonary fibrosis and chronic obstructive pulmonary disease (COPD), or who may experience postoperative pulmonary complications (PPCs) following surgery, undergo routine respiratory therapy to help stretch the muscles around the lungs, exercise the diaphragm, and loosen mucus build-up. One such routine respiratory therapy is spirometry, which utilizes a device called a respiratory therapy device to assess how well a patient's lungs are operating by measuring how much (volume) and how fast (flow) a patient can move air into and out of its lungs.

Incentive Spirometry devices used to perform spirometry are designed to mimic natural sighing or yawning by encouraging the patient to take long, slow, deep breaths, normally providing patients with visual or other positive feedback with a piston or a ball that moves inside a gauge. Incentive spirometry devices commonly include two different types—volume displacement devices and flow dependent devices.

The flow dependent incentive spirometry device is comprised of a casing consisting of three flow tubes containing light weight plastic ping-pong like balls, in which the casing is connected to a piece of tubing that has a mouthpiece at the end. The user inhales through the mouthpiece, which causes the pressure to drop within the casing and in turn causes the balls to rise in each of the flow tubes. Each tube is calibrated so that full displacement of the ping-pong like ball contained within equals a specific flow, which is indicated on the wall of the tube. The number of balls and the level to which they rise depends on the level of the flow achieved. At lower flows, the first ball rises to a level that depends on the magnitude of flow; with better flows, the second ball rises, and then the third ball. As flow exceeds the maximum allowance in the first tube, the ball in the second tube begins to rise until that tube is filled, and then the ball in the third tube rises. When using this device, a patient is instructed to hold the flow at the end so to keep the indicator balls elevated to full displacement for as long as possible.

The volume displacement respiratory therapy device allows a patient to inhale air through a mouthpiece and hose that is attached to a plastic bellows. As the patient inhales air through the hose, the bellow rises and an indicator on the device enclosure indicates the volumetric displacement. After the patient has achieved the maximum displacement, the patient is then told to hold the bellows in place for 5 to 10 seconds. After completion, the patient releases air and removes the mouthpiece, at which time the bellows return to their initial starting position.

However, both of these techniques often become strenuous and mundane for users with limited mobility, and patient compliance rates are low. Indeed, exercise capacity and tolerance are the most important factors in assessment of the clinical condition and prognosis of patients when conducting spirometry treatment. It can also be argued that the current respiratory therapy devices do not do enough to increase a user's overall tolerance to perform the exercise and maximize the benefits.

In addition to incentive spirometry, another common respiratory therapy device used to treat chronic respiratory diseases such as asthma is a metered dose inhaler (MDI). The MDI is used to administer bronchodilators and inhaled corticosteroids, both of which are used to treat asthma. Asthma is the most common chronic childhood disease in the United States, with a prevalence of approximately 8% of the youth population estimated by the CDC. Teaching children to properly use an MDI can be a challenge, especially with younger children, as it requires following a precise breathing sequence for proper dosage. For example, one study found that among children and adolescents who regularly use an MDI, only about a quarter were able to use the perfect technique, while almost half performed multiple steps incorrectly.

Even adults can struggle with proper inhaler use. A meta-analysis of inhaler use studies found that approximately 60% of adults prescribed to inhalers misuse the device, and that many don't receive sufficient training from their doctors. Without proper MDI technique, asthma sufferers do not receive the correct dosage of medication and may not get relief from their asthma symptoms. One deposition study found that while approximately 23% of the MDI medicine dose reached the lungs of the users with proper inhalation technique, only 7% of the dose reached the lungs of those using an improper technique. This decreased deposition can result in poor asthma control leading to increased hospitalizations, as well as the need for interventions, such as oral steroids, with a greater risk of serious side effects. These issues could be prevented if first step treatments were used properly.

An additional challenge with pediatric asthma management is tracking patient adherence to their prescribed treatment routines. One study of children aged 8-12 showed that they significantly over reported adherence to their inhaled corticosteroids when self-reporting (95% compliance) compared to electronic monitoring on their MDI devices (58% compliance); with those who did not comply being significantly more likely to experience exacerbations over the study period. Another study of children ages 7-12 found that both children and their parents' significantly over-reported MDI use, with parents over-reporting use even more than their children.

As such, a need exists for a new device, system and method for performing respiratory therapy, one that combines gaming and real-time feedback to guide users through the proper techniques when performing different forms of respiratory therapy, whether it involves the use of incentive spirometers or inhalers.

### SUMMARY OF THE INVENTION

The following summary of the invention is provided in order to provide a basic understanding of some aspects and features of the invention. This summary is not an extensive overview of the invention and as such it is not intended to particularly identify key or critical elements of the invention or to delineate the scope of the invention. Its sole purpose is to present some concepts of the invention in a simplified form as a prelude to the more detailed description that is presented below.

To achieve the foregoing and other aspects and in accordance with the purpose of the invention, a system and method for performing respiratory therapy is provided.

More specifically a device is provided that is attachable to any existing respiratory devices and/or form factors and provides gamification capabilities that utilizes gamification vastly increase the tolerance of a patient to utilize the device that in turn creates an overall better and more optimized healing method.

Accordingly, it is an object of the present invention to provide a new and improved system and method utilizing gamification to increase the likelihood of a user meeting therapy goals and thus creating an overall better and more effective therapy experience.

Another object of the present invention is to provide a new and improved device and system to perform respiratory therapy that utilizes advances in gaming technology to create a fun and immerse therapy session. In addition, rather than merely using conventional therapy methods, the system utilizes games created specifically to model the breathing patterns found in common respiratory therapy devices, with the goal of having users concentrate on the gaming aspect and not the therapy aspect. Structuring the therapy session in such a manner is important, as it will allow users to manifest a different and more positive perception of therapy, which is often seen as a physically challenging and demanding process, that in turn will create better and more effective therapy sessions.

Another object of the present invention is to provide an attachment member that utilizes wireless technology to increase the efficiency and effectiveness of known respiratory devices.

In exemplary embodiments, a processor-based respiratory device for respiratory therapy that combines gaming and real-time feedback to guide a user through proper respiratory techniques is described. The device comprises a housing having a hollow interior, an outer wall, an inlet, and an outlet, a radially inner portion defined by the housing to form a chamber, the chamber being positioned inside of the housing between the inlet and the outlet of the housing, the chamber being configured to allow air to flow from a user to a form factor when the user breathes into the chamber, at least a sensor positioned within the chamber and electronically coupled to the processor, wherein the sensor is configured to measure airflow within the chamber, a connection member positioned proximate the outlet, the connection member configured to form a seal with an opening of the form factor, wherein the processor comprises a communications interface coupled to a network, the communications interface being configured to output a signal to a graphical user interface based on the airflow in the chamber.

In exemplary embodiments, a system for respiratory therapy that combines gaming and real-time feedback to guide users through proper respiratory techniques is provided. The system comprises a substrate having a housing having a hollow interior, an outer wall, an inlet and an outlet; a radially inner portion defined by the housing to form a chamber, the chamber being positioned inside of the housing between the inlet and the outlet of the housing, the chamber being configured to allow air to flow from a user to a form factor when the user breathes into the chamber; at least a sensor positioned within the chamber and electronically coupled to the processor, wherein the sensor is configured to measure airflow within the chamber and the processor is communicably coupled to a network; a connection member positioned proximate the outlet, the connection member configured to form a seal with an opening of the form factor; a smart device that is in communication with the processor of the substrate over the network, wherein the smart device comprises: a graphical user interface; and a smart device processor in communication with the network; wherein the smart device comprises a mobile application configured to run software to allow the user to control a game that is played on the smart device using the substrate.

In exemplary embodiments, a method for respiratory therapy that combines gaming and real-time feedback to guide users through proper respiratory techniques is provided. The method comprises attaching a substrate to a form factor using a connection member positioned proximate an outlet of the substrate, the connection member configured to form a seal with an opening of the form factor; the substrate comprising at least one sensor and at least one processor having wireless communications protocol; connecting the substrate to a network; locating a smart device on the network, the smart device comprising a mobile application and a graphical user interface; outputting a signal from the substrate to smart device based on breathing of a user, wherein the signal corresponds to a proper way to breathe based on a user respiratory condition.

Other features, advantages, and aspects of the present invention will become more apparent and be more readily understood from the following detailed description, which should be read in conjunction with the accompanying drawings.

### BRIEF DESCRIPTION OF THE DRAWINGS

FIG. 1 is a simplified diagram of a respiratory therapy system utilizing gamification, in accordance with one embodiment of the present invention;

FIG. 2 is a side perspective view illustrating the internal components of the respiratory therapy device that may be used with the respiratory therapy system, in accordance with one embodiment of the present invention;

FIG. 3 is a perspective view of the gasket included on the respiratory therapy device, in accordance with one embodiment of the present invention;

FIG. 4 is a front perspective view of the respiratory therapy device that may be used with the respiratory therapy system, in accordance with one embodiment of the present invention;

FIG. 5 is a flowchart illustrating the flow of air in and out of the respiratory therapy device, in accordance with one embodiment of the present invention;

FIG. 6 is a front perspective view of a respiratory therapy device loaded with an inhaler for use with the respiratory therapy system, in accordance with one embodiment of the present invention;

FIG. 7 is a side perspective view of a respiratory therapy device loaded with an inhaler for use with the respiratory therapy system, in accordance with one embodiment of the present invention;

FIG. 8 is a perspective view of a respiratory therapy device connected to an incentive spirometer for use with the respiratory therapy system described herein, in accordance with one embodiment of the present invention;

FIG. 9 is a perspective view diagram illustrating the use of the respiratory therapy device system while connected to an incentive spirometer, in accordance with one embodiment of the present invention;

FIG. 10 is an exemplary game selection screen, in accordance with one embodiment of the present invention.

FIG. 11 is an exemplary feedback screen, in accordance with one embodiment of the present invention;

FIG. 12 is an example gameplay for the respiratory therapy system utilizing gamification, in accordance with one embodiment of the present invention;

FIG. 13 is an example gameplay for the respiratory therapy system utilizing gamification, in accordance with one embodiment of the present invention;

FIG. 14 an example gameplay for the respiratory therapy system utilizing gamification, in accordance with one embodiment of the present invention;

FIG. 15 an example gameplay for the respiratory therapy system utilizing gamification, in accordance with one embodiment of the present invention;

FIG. 16 is an exemplary results screen, in accordance with one embodiment of the present invention;

FIG. 17 is a diagram of the internal components of an optional embodiment of the respiratory therapy device, in accordance with one embodiment of the present invention;

FIG. 18 is a flowchart illustrating the flow of air in and out of the optional embodiment of the respiratory therapy device discussed in relation to FIG. 17, in accordance with one embodiment of the present invention;

FIG. 19 is a side perspective view of the optional embodiment of the respiratory therapy device discussed in relation to FIG. 17, in accordance with one embodiment of the present invention.

FIG. 20 is a perspective view of the optional embodiment of the respiratory therapy device being used by a user in accordance with one embodiment of the present invention.

FIG. 21 is a step-wise diagram illustrating a method for respiratory therapy in accordance with one embodiment of the present invention.

### DETAILED DESCRIPTION OF THE INVENTION

The present invention is best understood by reference to the detailed figures and description set forth herein.

Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described are shown. That is, there are numerous modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

It is to be further understood that the present invention is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

In one general aspect, the present invention is directed to computer-based systems and methods that utilize gamification to create a more immersive, efficient and effective therapy treatment for individuals required to use an respiratory therapy device, with each therapy game being specifically tailored to account for an individual's health needs and its prior performance on each therapy game.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be also understood to refer to functional equivalents of such structures. The present invention will now be described in detail with reference to embodiments thereof as illustrated in the accompanying drawings.

As used herein, the term “substrate” shall mean an add-on device that is attachable or connectable to a plurality of form factors, including but not limited to respiratory devices such as inhalers, spirometers nebulizers, Positive Expiratory Pressure (PEP) devices and the like. As used herein, the device 102 may be referred to as a “substrate”.

As used herein, the term “user” shall mean any individual who uses the system to perform respiratory therapy or to otherwise aide their breathing issues. More specifically, a user will either be a patient who has been prescribed physical therapy or an individual who is seeking an in-home therapy treatment to help with lung and breathing related health issues.

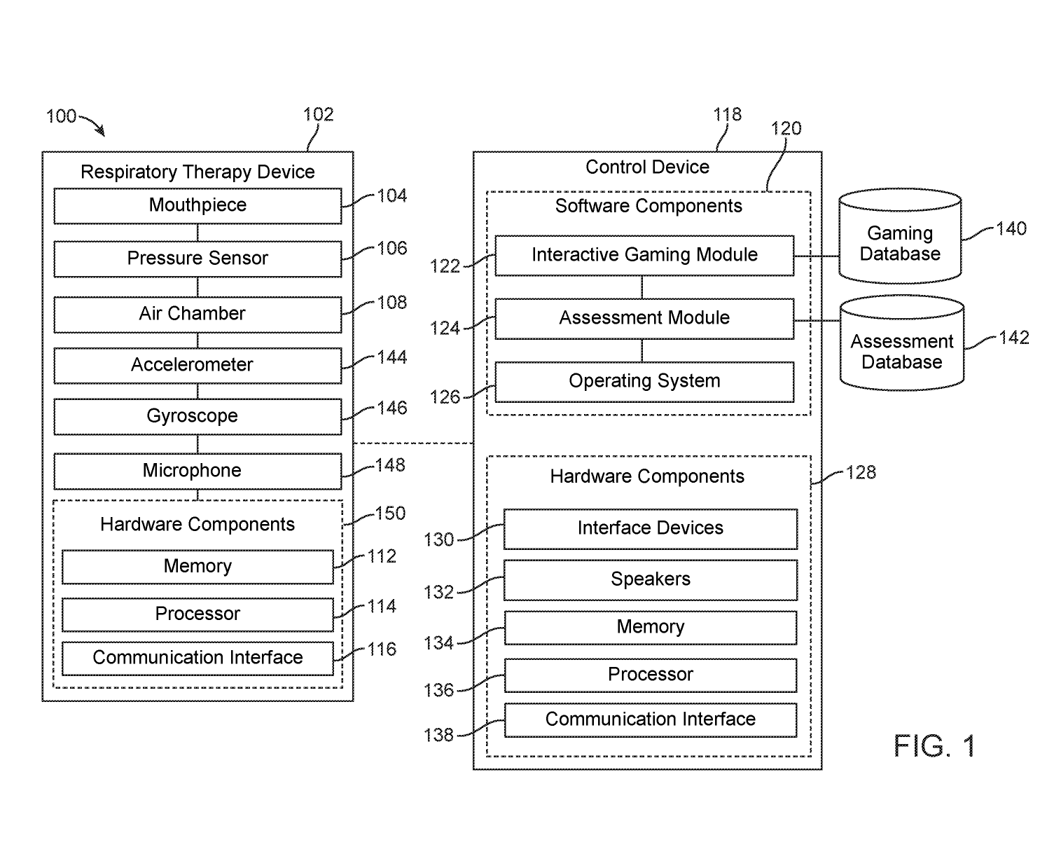

Referring now to FIG. 1, a diagram of a respiratory therapy system utilizing gamification in accordance with one embodiment of the present invention, is presented generally at reference numeral 100. The embodiment 100 is a simplified example of a therapy environment in which a respiratory therapy device 102 may operate with a smart device 118. The smart device may in the form of an application (e.g., downloadable mobile application) that is downloadable on to a smartphone, tablet, television, or any smart device that comprises a graphical user interface (GUI). The embodiment 100 illustrates the functional components of a system. The system may comprise the device (substrate), network, operating system of a smart device, and the GUI of said smart device. In some embodiments, the functional component may be a hardware component, a software component, or a combination of hardware and software. Some of the components may be application level software, while other components may be operating system level components. In some cases, the connection of one component to another may be a close connection where two or more components are operating on a single hardware platform. In other cases, the connection may be made over network connections spanning long distances and a plurality of nodes. Each embodiment may use different hardware, software, and interconnection architectures to achieve the described functions.

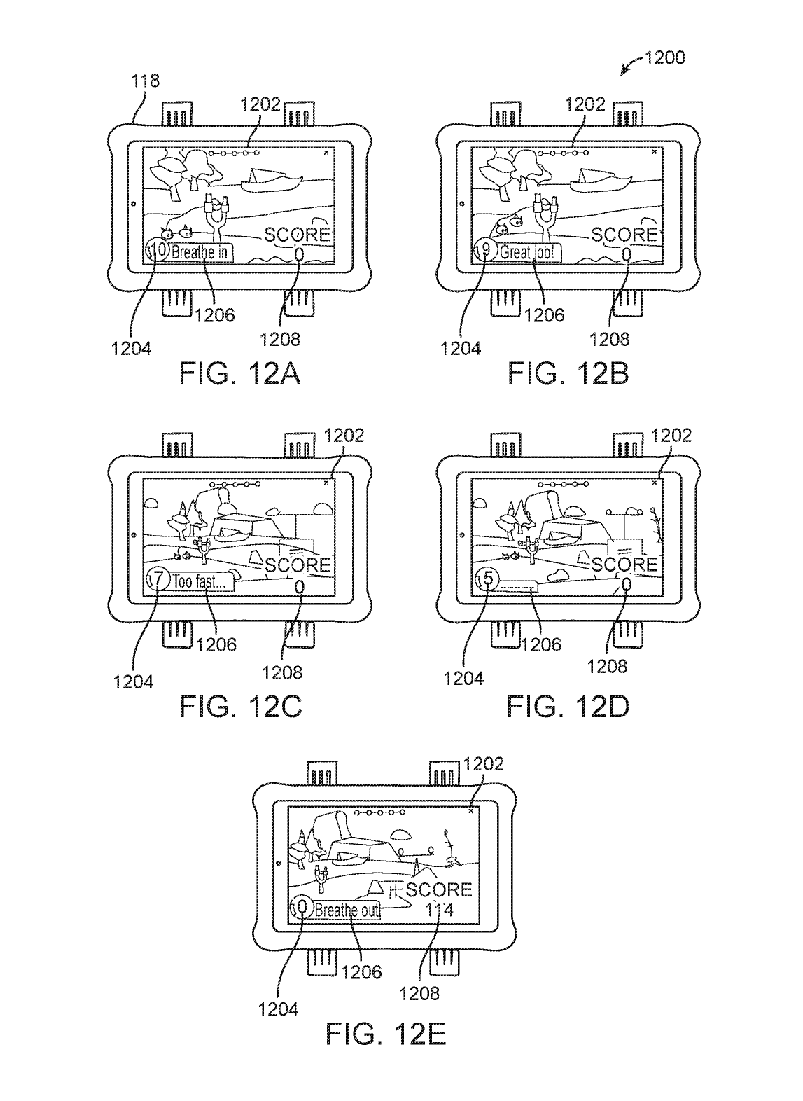

Still referring to FIG. 1, the respiratory therapy device or substrate 102 comprises a mouthpiece 104, a pressure sensor 106, and an air chamber 108. In an embodiment, the mouthpiece 104 is an opening coupled to the respiratory therapy device 102 though which a user may perform certain breathing maneuvers. The air chamber 108 is provided to allow for air to flow in and out of the respiratory therapy device 102. As discussed further herein in relation to FIGS. 12-15, in operation, a user inhales and exhales breaths of air through the mouthpiece 104 based upon specific instructions provided by the smart device 118, with the air chamber 108 allowing the air to travel in and out of the therapy device. In addition to the mouthpiece 104, at least one pressure sensor 106 is coupled to the hardware components 150 disposed within the respiratory therapy device 102. In the current embodiment, a silicon pressure sensor is utilized and has a standard error of 2.5% over the temperature compensated range between +10° C.+60° C. and a pressure range of −0.3 PSI to 0.3 PSI at a voltage output of 0.5V to 4.5V, however, any type of pressure sensor that provides accurate measurement may be used.

In optional embodiments, additional sensors may be employed to provide different or additional functionality to the device. As an example, in optional embodiments, an accelerometer 144, gyroscope 146 or microphone 148 may be used. Each of the additional sensors may provide additional gaming capabilities including but not limited to: Utilizing direction and movement of the device and relaying that movement to the GUI and for providing vocal communication ability with the game itself or others playing the game. In further optional embodiments, rotary sensor and linear sensors (e.g., potentiometers) may be utilized.

Still referring to FIG. 1, in the current embodiment, a sensor to measure airflow is not utilized. However, in optional embodiments, a separate sensor to measure airflow may be integrated into the respiratory therapy device 102. The ability to measure airflow is important because it allows the assessment module 124 to analyze a user's breathing to determine their level success with the game it played as well as to create follow-up gameplay that will allow a user to build off of its prior session to allow for a more effective and efficient therapy practice. This may incorporate machine learning so that the system improves over time with respect to each individual's needs. In the current embodiment, we use the pressure readings collected by the pressure sensor 106 to calculate the airflow utilizing the following equation:

$$V = \sum {F}_{t} \times \frac{1}{f}$$

Utilizing this formula, we can calculate the flow and volume relationship utilizing the pressure readings collected from the pressure sensor 106. Furthermore, based on the following formula, we also know that volume of air is proportional to the integral of the flow over time:

$$F = \frac{Δ P}{R} = \frac{( {P}_{A} - {P}_{V} )}{R}$$

With this information, we are able to calculate the vital capacity (VC), forced expiratory volume at 1 second (FEV1), and the peak expiratory flow (PEF) from a single breath curve. We can determine these values from a single breath curve representing a user exhaling maximally and forcibly, similarly to the airflow through a standard spirometer. The VC is presented by the entire integral under the curve, multiplied by a constant, K. The FEV1 is represented by the integral under the curve at one second over the integral of the entire curve (as a percentage), and PEF would be the relative maximum airflow value of the curve, multiplied by a constant, K.

Still referring to FIG. 1, the respiratory therapy device 102 may also comprise hardware components 150 that are in communication with, and coupled to, the pressure sensor 106. In exemplary embodiments, the hardware components 150 may comprise a random access memory unit 112, a central processing unit 114 and a communication interface 116. The random access memory 112 may store executable code as well as data that may be immediately accessible to the processor 114, while the communication interface 116 may include wireless interfaces through which the respiratory therapy device 102 may communicate with other devices. In the current embodiment, the communication interface 116 is coupled to a wireless Bluetooth® communication interface to allow for the respiratory therapy device 102 to communicate with the smart device 118. In additional embodiments, the communication interface 116 may utilize hardwired micro-USB connection or other forms of wireless communication such as WiFi or Bluetooth® to allow the respiratory therapy device 102 to connect with the smart device 118.

Still referring to FIG. 1, the respiratory therapy device 102 is communicatively coupled to a smart device 118 via their communication interfaces, 116 and 138, respectively. In the exemplary embodiments, the respiratory therapy device 102 is communicatively coupled to a smart device 118 through the use of a Bluetooth connection. In optional embodiments, the respiratory therapy device 102 may be communicatively coupled to a smart device 118 through the use of a hardwired micro-USB connection or other forms of wireless communications such as WiFi or Bluetooth®. The smart device 118 may represent the architecture of a computing device. In some embodiments, the smart device 118 may be a personal computer, network appliance, interactive kiosk or other device. The smart device 118 may also be a portable device, such as a tablet, laptop computer, netbook computer, personal digital assistant, mobile telephone, or other mobile device. In the current embodiment, the smart device 118 is a portable device in the form of a tablet, but in optional embodiments the smart device 118 may be a mobile phone, laptop or desktop computer, television, or any digital interface.

Still referring to FIG. 1, the smart device 118 may comprise multiple hardware components 128 such as interface devices 130, speakers 132, random access memory 134, a central processing unit 136, and a communication interface 138. In exemplary embodiment, the interface devices 130 may include monitors, displays, keyboards, pointing devices, and any other type of user interface device. The random access memory 134 may store executable code as well as data that may be immediately accessible to the processor 136. In the current embodiment, the random access memory 134 is used to store any data needed to play the games.

Still referring to FIG. 1, the communication interface 138 may also include hardwired and wireless interfaces through which the smart device 118 may communicate with other devices. In the current embodiment, the smart device 118 utilizes its communication interface 138 to communicate with the respiratory therapy device 102 via wireless Bluetooth® technology. The communication interface 138 also allows the smart device 118 to communicate with certain other remote access devices, which may include any off-site device used by a physician or therapy provider to review results and set parameters for new therapy sessions. In optional embodiments, the communication interface 138 may also utilize wired technology or other wireless technology to allow for communication with the respiratory therapy device 102.

Still referring to FIG. 1, in the present embodiment the smart device 118 may also comprise software components 120 such as an operating system 126, which resides on a non-transitory media on which various applications may be executed, as well as an interactive gaming module 122 and an assessment module 124. The interactive gaming module 122 allows for the operation of any of the therapy games which the interactive gaming module 122 sources from the gaming database 140 it is coupled to. The gaming database 140 is configured to store all of the therapy games, which will be discussed later with reference to FIGS. 12-15. In exemplary embodiments, the gaming database 140 can be updated via use of the communication interface 138, whereby the user can connect to a remote device such as a third-party storage device or to a virtual shop where new games can be stored to its gaming database 140 and played by a user via the interactive gaming module 122.

Still referring to FIG. 1, the software components 120 may also comprise an assessment module 124, which is used to analyze and score a user's performance on any game played via the interactive gaming module 122, which is coupled to an assessment database 142. The user may input its base scores within the assessment module 124 prior to performing its first gaming session. After the performance of each gaming session thereafter, the scores are stored in the assessment database 142, and may be recalled by the interactive gaming module 122 to make user specific adjustments to a game to account for the user's therapy performance. The scores stored in the assessment database 142 may also be accessed by third parties such as therapy providers and doctors via use of the communication interface 138 who may use the assessments to modify the specific user's games in the gaming database 140 to maximize the effectiveness and efficiency of a particular therapy session, thus allowing for both in-person and remote therapy assessments. The communication interface may employ HIPPA compliant security.

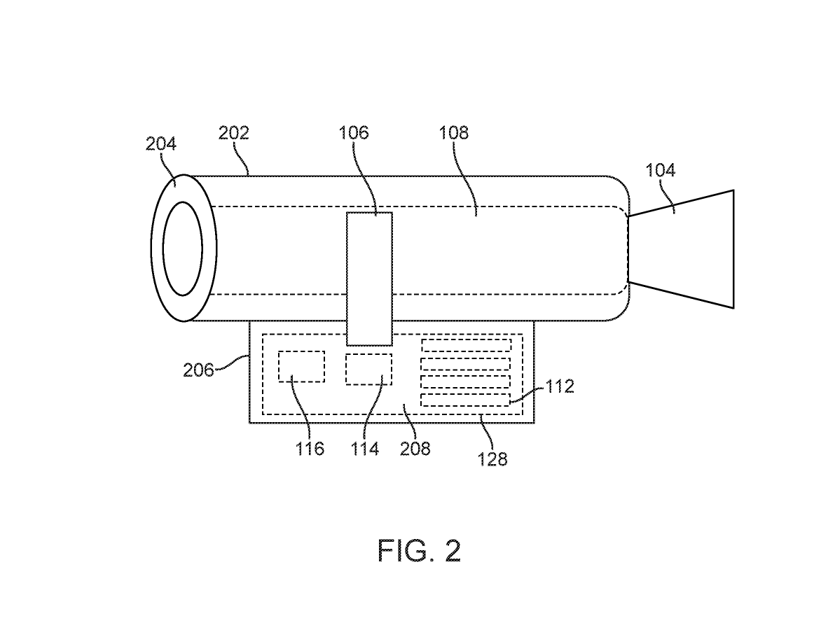

Referring now to FIG. 2, a side perspective view illustrating the internal components of the respiratory therapy device that may be used with the respiratory therapy system in accordance with one embodiment of the present invention is presented generally at 200. The respiratory therapy device 102 consists of a main body or housing 202, the housing having a hollow interior, an outer wall, an inlet and an outlet, such that the radially inner portion defined by the housing to form a chamber 108, the chamber 108 being positioned inside of the housing between the inlet at the mouthpiece, and an outlet near a gasket 204. The air chamber 108, gasket 204, and mouthpiece 104 are coupled to the main body 202, a lower compartment 206, where the hardware components 108 are stored, as well as a pressure sensor 106.

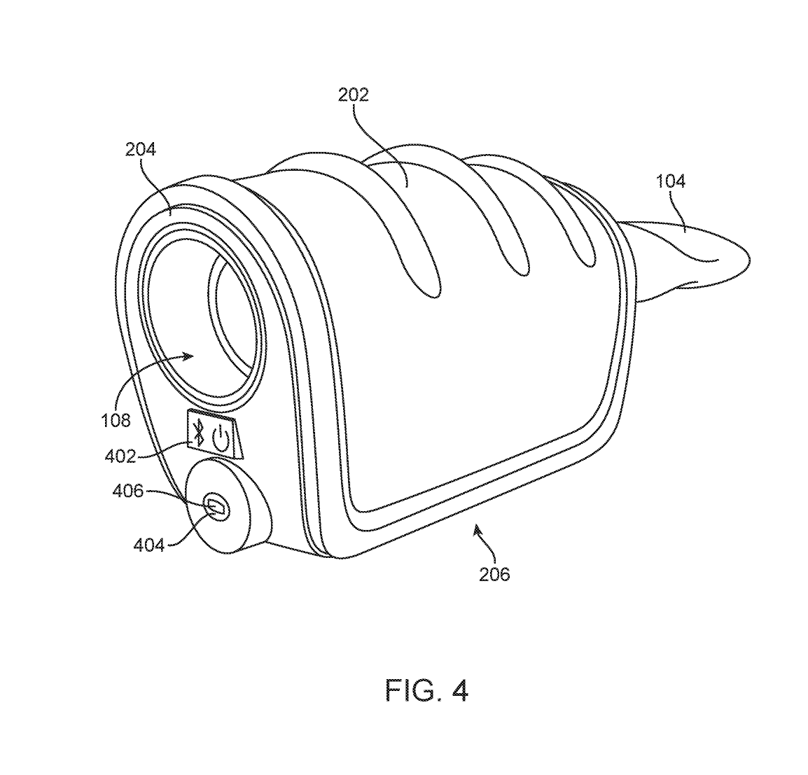

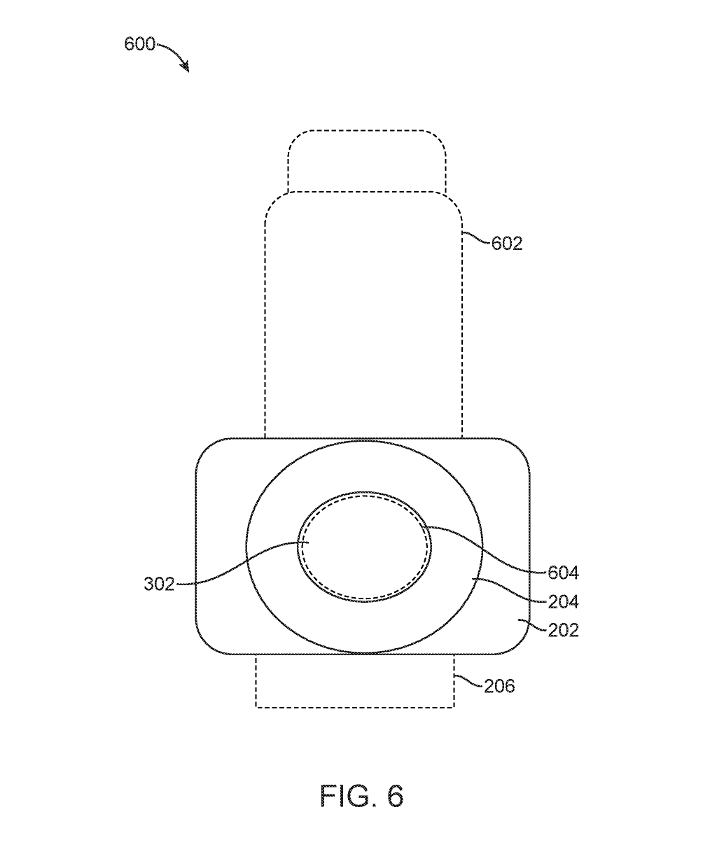

Still referring to FIG. 2, the main body 202 and lower compartment 206 are designed with grooves shaped to conform to a human hand such that a user may grip the respiratory therapy device 102 in the most natural position; this will in turn allow for a user to focus on the actual performance of the therapy games and less on its use of the respiratory therapy device, thereby increasing the chance of having a more effective and efficient therapy session. It should be noted that while the hardware components are shown in a compartment 206, they may be disposed in any part of the housing, such as is shown in FIG. 4. The gasket 204 allows for attachment to a plurality of form factors (i.e. respiratory therapy devices) such as incentive spirometers and inhalers to be coupled creating the ability for these preexisting form factors to be used to perform the system and method of respiratory therapy utilizing gamification described herein. The use of other respiratory devices to perform the system and method provided for herein is further discussed in relation to FIGS. 6-9.

Still referring to FIG. 2, the mouthpiece 104 allows the user to inhale and exhale air, which in operation allows for playing of the respiratory therapy games. The air chamber 108 serves as a port through which air may flow in and out of the respiratory therapy device 102. The flow of air and its relation to playing a respiratory therapy game is further discussed in relation to FIGS. 12-15. The pressure sensor 106 is located within both the air chamber 108 and lower compartment 206, and is used to calculate the airflow and analyze a user's performance, thereby allowing for modifications to the user's games to create a more effective and efficient therapy session. In optional embodiments, it may be in a single compartment in communication with chamber and processing circuitry and configured to send signals to the GUI based on respiratory patterns of the user.

Still referring to FIG. 2, the lower compartment 206 houses the hardware components 110. The hardware components 110 comprise random access memory 112, a central processing unit 114, and a communication interface 116, all of which is coupled to a circuit board 208. In the current embodiment, the communication interface 116 includes a wireless interface through which the respiratory therapy device 102 may communicate with other devices. In the current embodiment, the communication interface 116 utilizes Bluetooth® to allow the respiratory therapy device 102 to communicate with the smart device 118. In optional embodiments, the communications interface 116 may include hardwired interfaces such as micro-USB and USB, or may utilize other wireless technologies such as WiFi, to communicate with the smart device 118. The random-access memory 112 may store executable code as well as data that may be immediately accessible to the processor 114. The data stored in the random-access memory may include the games and performance scores, which are further discussed in regard to FIGS. 12-15.

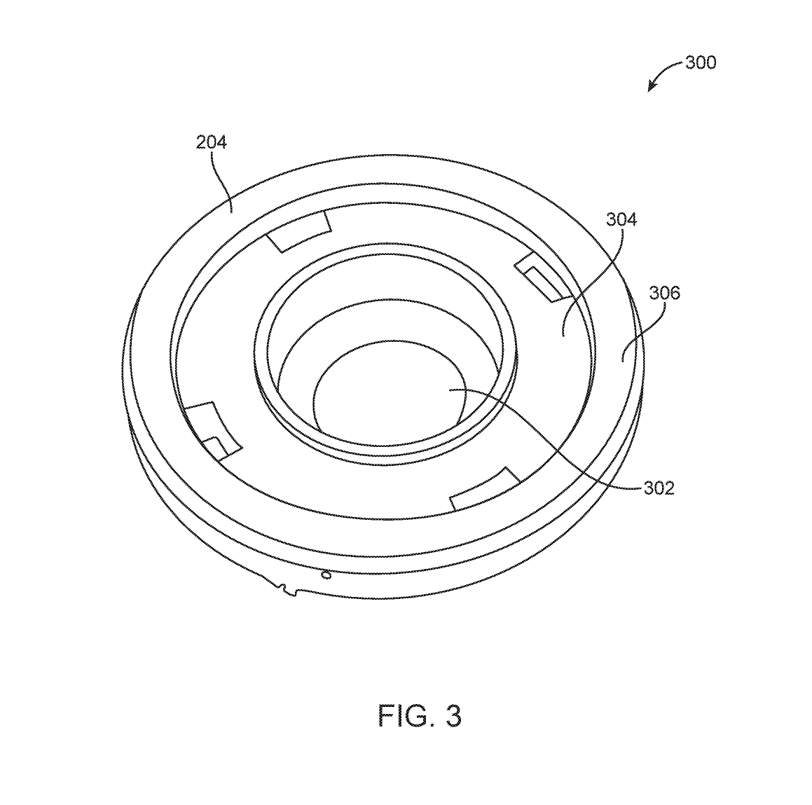

Referring now to FIG. 3, a perspective view of a connection member or gasket 204 on the respiratory therapy device in accordance with one embodiment of the present invention is presented generally at 300. The gasket 204 comprises an attachment opening 302 that allows for other respiratory devices to be coupled to the respiratory therapy device of the present invention, and comprises multiple tiers 304 and 306. The gasket 204 is coupled to the rim of the air chamber 108 to ensure that a complete seal is made between a user inserted respiratory therapy device (such as an incentive spirometer or metered dose inhaler) and the attachment opening 302, allowing for proper operation of the respiratory therapy device 102. If the form factor opening is large, then the second tier 304 may provide the seal, and if it is larger still, the third tier 306 may provide the seal. The gasket may further comprise a sensor 308 that is configured to sense when a form factor (e.g., metered-dosed inhaler) is activated based on sensing a user shaking the device or other activation step. The gasket sensor 308 may be optionally configured to sense when the device attaches to a form factor, and intuitively connect to the smart device 118, which will in turn pull up the gaming menu via wireless communication such as Bluetooth®. In this way, the device is able to be used with a myriad of existing respiratory devices or form factors, such as common inhalers, spirometers, and the like. In optional embodiments, each tier may be made from sufficiently pliable or elastic material that stretches, expands, and contracts to create a seal between the substrate 102 and the form factor.

Referring now to FIG. 4, a front perspective view of the respiratory therapy device that may be used with the respiratory therapy system in accordance with one embodiment of the present invention, is presented generally at 400. In this embodiment, the main body 202, lower compartment 206, gasket 204, air chamber 108 and mouthpiece 104 are shown. In this embodiment, the Bluetooth® or wireless communication control button 402 is shown together with power button 404 and wired communication port 406.

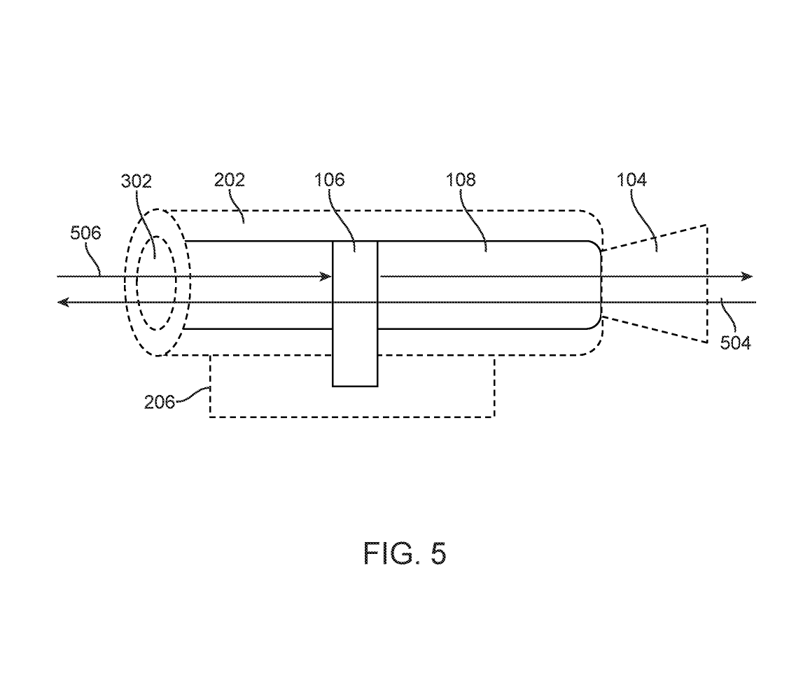

Referring now to FIG. 5, a flowchart illustrating the flow of air in and out of the respiratory therapy device in accordance with one embodiment of the present invention, is presented generally at 500. The pressure sensor 106 is located within both the main body 202 and in some embodiments, in lower compartment 206. Depending on the in-game instructions provided on the smart device 118, the user will breathe air in and/or out of the respiratory therapy device 102 utilizing the mouthpiece 104. When the user breathes air into the respiratory therapy device 102, meaning the user exhales into the respiratory therapy device 102, the air will travel through the mouthpiece 104 and the air chamber 108 into the pressure sensor 106, where the pressure reading is calculated. Thereafter, the air then flows out of the air chamber 108 via the attachment opening 302. The flow of air is shown at 504. If the user is required to breathe air out of the respiratory therapy device 102, meaning the user inhales air out of the respiratory therapy device 102, the air will travel into the air chamber 108 through the attachment opening 302, pass through the pressure sensor 106 and then be directed back into the air chamber 108, out through the mouthpiece 104, and into the user's mouth. The flow of air when a user inhales air out of the respiratory therapy device 102 is shown as 506.

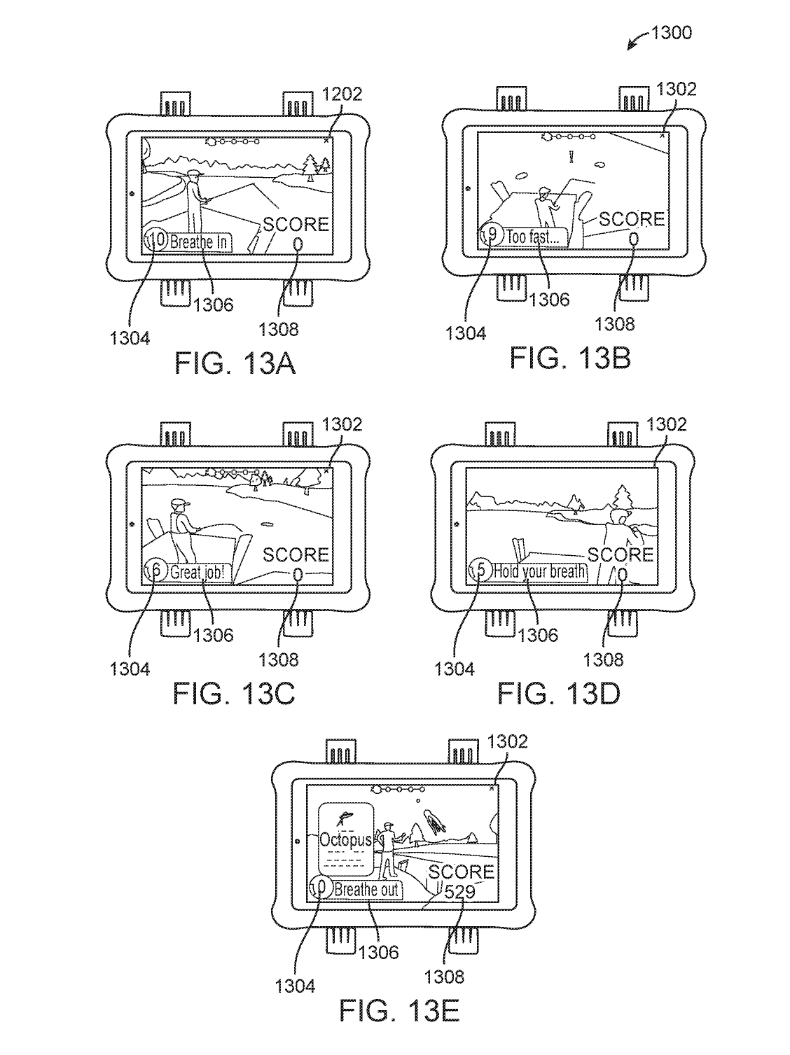

Referring now to FIG. 6, a front perspective view of the respiratory therapy device loaded (i.e., substrate) with a metered-dosed inhaler (i.e., MDI form factor) for use with the respiratory therapy system in accordance with one embodiment of the present invention, is presented generally at 600. In this embodiment, the inhaler 602 is coupled to the respiratory therapy device 102 by inserting the inhaler's outlet 604 into the attachment opening 302 located on the gasket 204. The specific game play that would be performed by a user while utilizing the respiratory therapy device loaded with an inhaler shall be discussed in reference to FIG. 13.

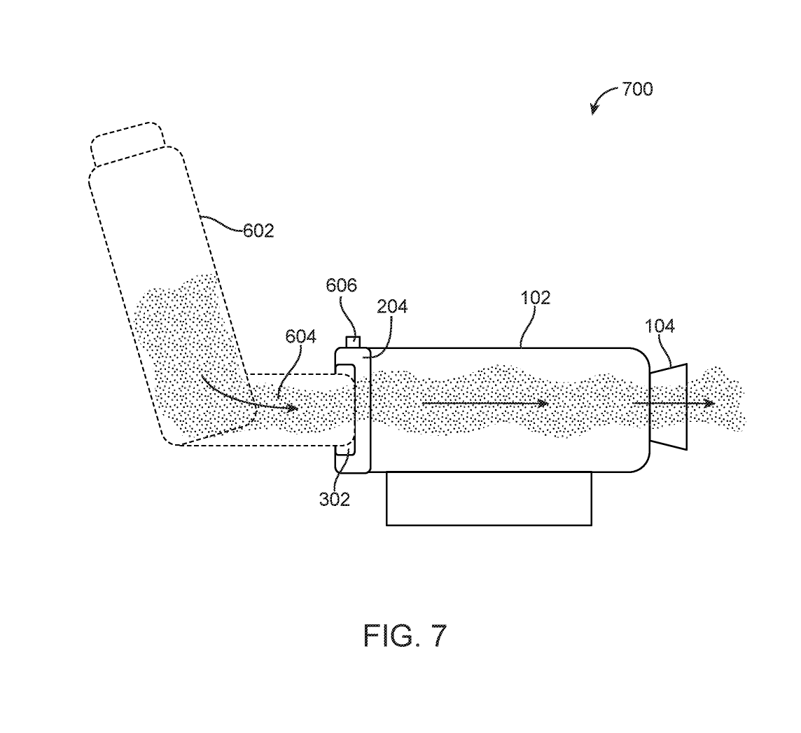

Referring now to FIG. 7, a side perspective view of the respiratory therapy device loaded with a metered-dosed inhaler (MDI) for use with the respiratory therapy system in accordance with one embodiment of the present invention, is shown generally at 700. In this embodiment, the inhaler 602 is inserted into the attachment opening 302 located within the gasket 204 coupled to the respiratory therapy device 102. In this embodiment, the mouthpiece 104 is shown for reference. The specific game play that would be performed by a user while utilizing the respiratory therapy device loaded with an inhaler shall be discussed in reference to FIG. 13.

Specifically with relation to MDIs, the device 102 serves as a breath controlled “game controller”, wherein inhalations and exhalations can trigger gaming actions allowing users to play games on the smart device 118. The games are configured to guide them through the proper MDI technique while also tracking usage. In this way, users are trained to model their inhalation after the ideal breathing pattern for MDI use, resulting in improved aerosol delivery and improved patient compliance when compared to standard MDI and spacer combinations.

In optional embodiments, the pressure sensor 106 is configured to sense when the MDI is activated, that is, when shakes the device to prepare for dosing, also detect the user begins to release a dose, and also detected the user's inhalation and exhalation through the MDI and into the device 102 while providing real time feedback via GUI to the user.

In optional embodiments, the accelerometer 144, gyroscope 146 or microphone 148 may be configured to detect activation of the MDI, and detect the user's inhalation and exhalation though the MDI.

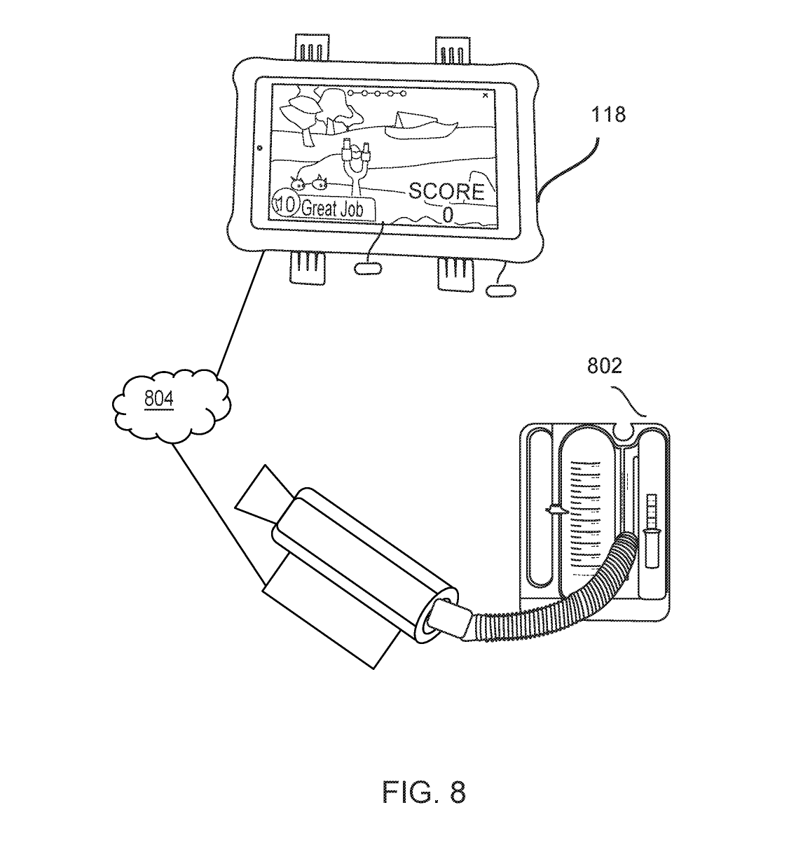

Referring now to FIG. 8, a perspective view of a respiratory therapy device connected to an incentive spirometer (i.e., another form factor) for use with the respiratory therapy system described herein in accordance with one embodiment of the present invention, in shown generally at 800. In this embodiment, the incentive spirometer 802 is shown coupled to the respiratory therapy device 102. The hose of the incentive spirometer 802 is coupled to the respiratory therapy device 102 via the attachment opening 302 located on the gasket 204. In this embodiment, the mouthpiece 104 is shown for reference.

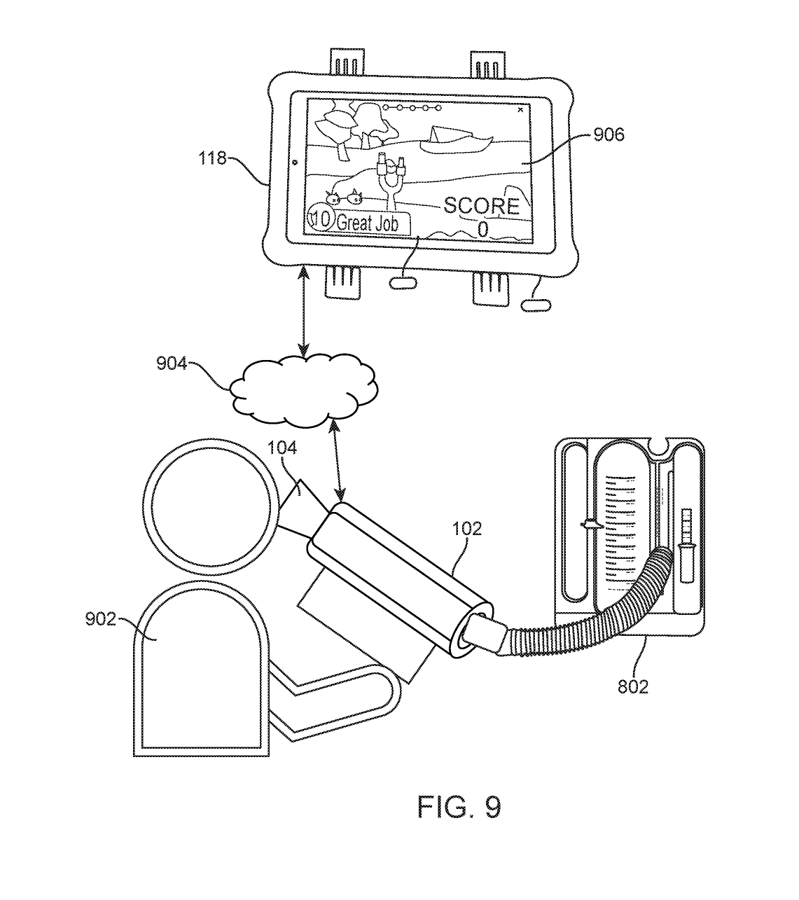

Referring now to FIG. 9, a perspective view illustrating the use of the respiratory therapy gaming system along with an incentive spirometer in accordance with one embodiment of the present invention is presented generally at 900. The incentive spirometer 102 is coupled to the respiratory therapy device 802. The user 902 is depicted holding the respiratory therapy device 102 to their mouth coupled to the mouthpiece 104. The respiratory therapy device 102 is connected to the smart device 118 via a local network 904 through the use of wireless Bluetooth® technology. The game the user is playing would be displayed on the screen 906. The specific game play shall be discussed in reference to FIGS. 12-14.

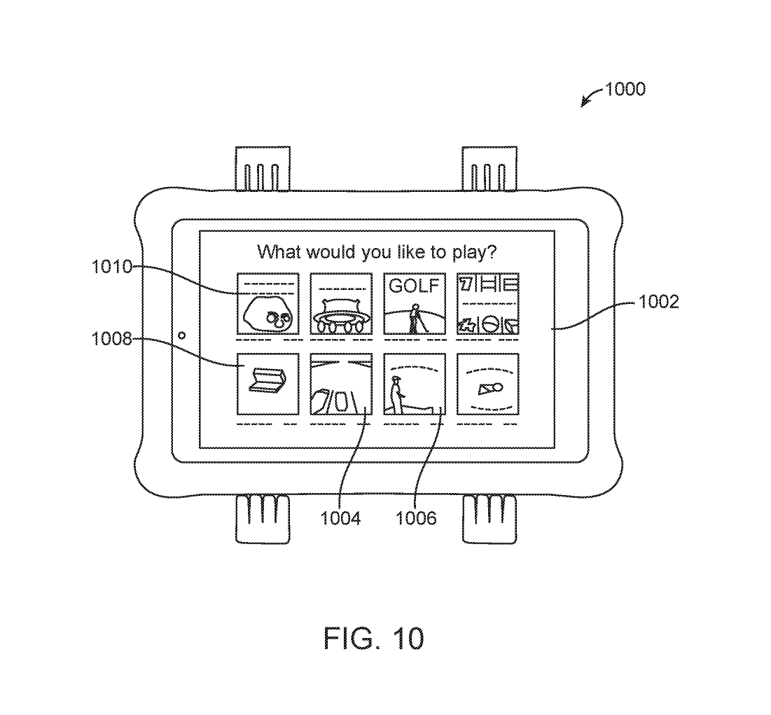

Referring now to FIG. 10, an exemplary game selection screen in accordance with one embodiment of the present invention, is presented generally at 1000. In current embodiments, a user would see the game selection screen 1002 when it either first logs into therapy application located on the smart device 118 or when it completes one therapy game and elects to play another game. In the current embodiment the game selection screen 1002 may display all of the available therapy games that the user may participate it. In optional embodiments, the game selection screen 1002 may include a link that allows the user to visit a third-party external location (e.g., website or application store) where it may purchase additional games.

Still referring to FIG. 10, in embodiments, each game may be correlated with a specific respiratory conditions. As an example, game 1004 may be configured for cystic fibrosis, game 1006 may be configured for asthma, game 1008 may be configured for COPD, and game 1010 may be configured for pneumonia. Each game creates a different incentive breathing pattern that best matches a user's level of need and experience with performing respiratory therapy. Furthermore, the system is designed based on breathing patterns found to have remedial effects to that particular condition.

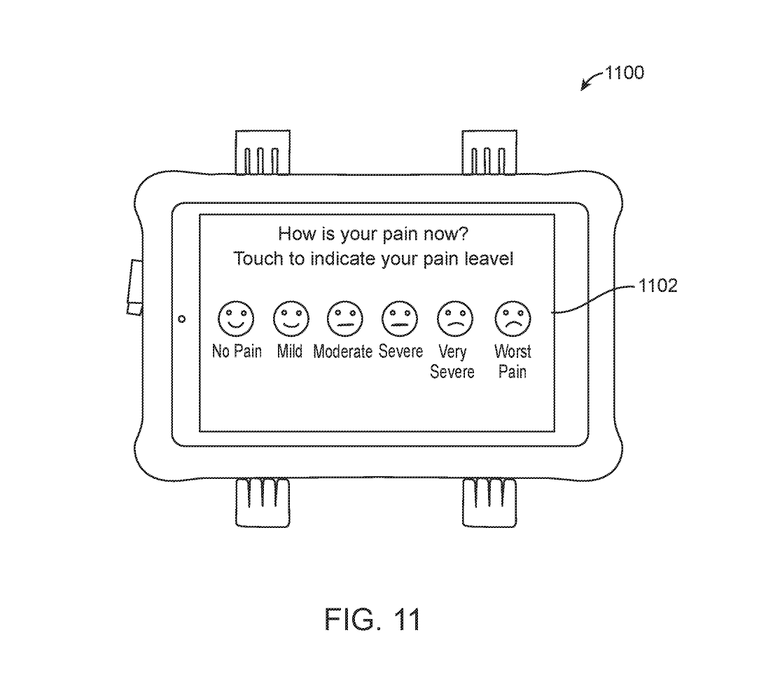

Referring now to FIG. 11, an exemplary feedback screen or GUI in accordance with one embodiment of the present invention is presented generally at 1100. A user would see this pain rating selection screen 1102 before he or she plays a therapy game as well as after he or she completes a therapy game. The pain rating selection screen 1102 includes a very easy to understand rating system, going from “none” to “mild” to “moderate” to “severe” to “very severe” to “worst pain.” In the current embodiment, the user would select which rating best describes their pain feeling prior to playing the appropriate therapy game. Once the game is completed, the user is again prompted with this screen and must select which rating best describes their pain feeling after completing the therapy game. These responses are logged and saved to the assessment database 142 and are transmitted via HIPPA compliant means and used by the user's therapist to alter the therapy sessions to ensure an effective and efficient therapy practice. In optional embodiments, machine learning technology may be implemented to perform automated adjustments to the therapy game based upon the level of pain the user experiences upon completion of the game as compared to the level of pain that the user was experiencing prior to playing the game.

Referring now to FIGS. 12-15, example gameplay for the respiratory therapy system utilizing gamification in accordance with one embodiment of the present invention are presented generally at 1200, 1300, 1400, and 1500. In exemplary embodiments, the games 1200, 1300, 1400, and 1500 are all 10 seconds in duration. Generally speaking, the games 1200, 1300 and 1400 begin with a user breathing in at the 10 second mark; at the 5 second mark the user is instructed to hold its breath; and at the 0 second mark the user is instructed to breathe out. Of course, different play can be utilized depending upon the respiratory condition. Game 1500 is specifically designed for use with an inhaler and while the game still begins at the 10 second mark, a different breathing pattern is used to best teach individuals how to use the inhaler. Each of the examples will now be discussed separately, as each one comprises a different game format. The games incentives a user to breathe in a certain pattern that is commissure with a certain disease, the breathing pattern being configured to alleviate the conditions of the disease and/or obviate the disease.

### Example 1

In FIG. 12, a user is playing physics puzzle-type game (the game “type” is well-known via Angry Birds®). In the current embodiment, the in-game screen 1202 on the smart device 118 comprises a counter 1204, score display 1208, and a status indicator 1206. In optional embodiments, the in-game screen 1202 may comprise other items. In FIG. 12A, the user is seeing the first screen of gameplay, indicated by the counter 1204 being at “10,” which is in the form of seconds, and the status indicator 1206 prompting the user to “breathe in.” In the beginning, the score display 1208 is at “0.” In FIG. 12B, the user begins the game. The counter 1204 is now at “9,” which indicates 9 seconds remain in the game. The status indicator 1206 notifies the user that they are doing a “great job” and therefore, they should continue to perform the step indicated in the first screen of gameplay, which was to breath in, as they have been. The user's score display 1208 remains at “0.” In FIG. 12C, the status indicator 1206 notifies the user that it is breathing in “too fast.” In response, the user should slow its breathing. The counter 1204 is now at “7,” which indicates that 7 seconds remain in the game. The user's score display 1208 remains at “0.” In 10D, the status indicator 1206 notifies the user to “hold your breath,” and in response, the user should hold its breath. The counter 1204 is now at “5,” which indicates that 5 seconds remain in the game. In 12E, the user is now at the end of the game. The counter 1204 is at “0,” indicating there is no time left in the game. At this point, the status indicator 1206 instructs the user to “breathe out,” which causes the game to play out and for the user to obtain points. The score display 1208 now indicates a score of “114.”

### Example 2

In FIG. 13, a user is playing a fishing game. In the current embodiment, the in-game screen 1302 on the smart device 118 comprises a counter 1304, score display 1308, and a status indicator 1306. In optional embodiments, the in-game screen 1308 may comprise other items. In FIG. 13A, the user is seeing the first screen of gameplay, indicated by the counter 1304 being at “10,” which is in the form of seconds, and the status indicator 1306 prompting the user to “breathe in.” In the beginning the score display 1308 is at “0.” In FIG. 13B, the user begins the game. The counter 1304 is now at “9,” which indicates that 9 seconds remain in the game. The status indicator 1306 notifies the user that they are going “too fast” and therefore, they should adjust their breathing to be slower. The user's score display 1308 remains at “0.” In FIG. 13C, the status indicator 1306 notifies the user that it is doing a “great job,” indicating that the user accurately adjusted its breathing based upon the status indicator 1306 message in FIG. 13B. The counter 1304 is now at “6,” which indicates 6 seconds remain in the game. The user's score display 1308 remains at “0.” In FIG. 13D, the status indicator 1306 notifies the user to “hold your breath,” and in response, the user should hold its breath. The counter 1304 is now at “5,” which indicates that 5 seconds remain in the game. The score display 1308 remains at “0.” In FIG. 13E, the user is now at the end of the game. The counter 1304 is at “0,” indicating there is no time left in the game. At this point, the status indicator 1306 instructs the user to “breathe out,” which causes the game to play out and for the user to obtain points. The score display 1308 now indicates a score of “529.” Optionally, this game will be directed to the proper use of an MDI. In this way, the game begins by prompting the user to removing the cap and shake the device, which will be measured via an on-board accelerometer. The user will then be instructed to: Put their mouth on the device, take a full breath in and then exhale, “puff” the medicine while inhaling slowly, and then, to hold their breath for 5-10 seconds before releasing. This enables the device to pick up on all inhalations and exhalations, as well as the actual puff of the inhaler, and ensures that the user does not move on to the next step until they have completed the previous step. Users, as well as their doctors and parents, will also be able to view their past use history on their smart device, including time of last dosage.

### Example 3

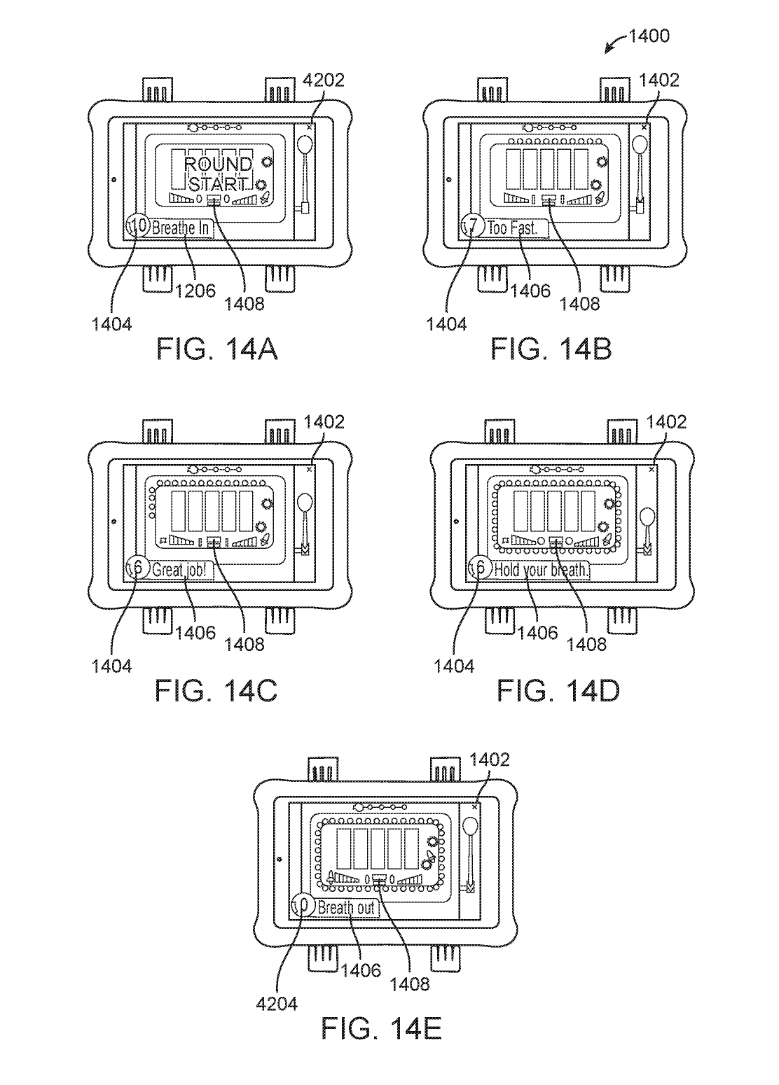

In FIG. 14, a user is playing a casino game. In the current embodiment, the in-game screen 1402 on the smart device 118 comprises a counter 1404, score display 1408, and a status indicator 1406. In optional embodiments, the in-game screen 1402 may include other items. In FIG. 14A, the user is seeing the first screen of gameplay, indicated by the counter 1404 being at “10,” which is in the form of seconds, and the status indicator 1406 prompting the user to “breathe in.” In the beginning the score display 1408 is at “0” and will remain at “0” until the user completes the game and a score is assessed. In FIG. 14B, the user begins the game. The counter 1404 is now at “7,” which indicates that 7 seconds remain in the game. The status indicator 1406 notifies the user that they are going “too fast” and therefore, they should adjust their breathing to be slower. In FIG. 14C, the status indicator 1406 notifies the user that it is doing a “great job,” indicating that the user accurately adjusted its breathing based upon the status indicator 1406 message in FIG. 14B. The counter 1404 is now at “6,” which indicates six seconds remain in the game. The user's score display 1408 remains at “0.” In FIG. 14D, the status indicator 1406 notifies the user to “hold your breath,” and in response, the user should hold its breath. The counter 1404 is now at “5,” which indicates that 5 seconds remain in the game. The score display 1408 remains at “0.” In FIG. 14E, the user is now at the end of the game. The status indicator 1406 is at “0,” indicating there is no time left in the game. At this point, the status indicator 1406 instructs the user to “breathe out,” which causes the game to play out and for the user to obtain points. The score display 1408 now indicates a score of “980.”

### Example 4

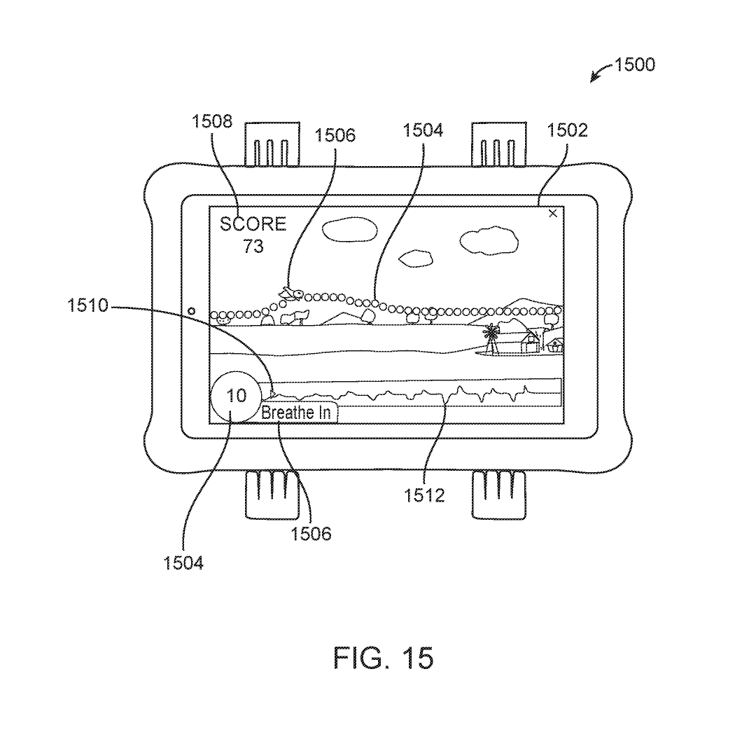

In FIG. 15, a sample game is provided that is specifically designed to be used for clearing congestion and drainage secretion. Unlike the other games discussed in regard to FIGS. 12-14, this game operates only using a “breathe in” command. FIG. 15 displays the screen the user would see when it first begins the game. In the current embodiment, the in-game screen 1502 on the smart device 118 comprises a counter 1504, score display 1508, status indicator 1506, optimal breath path 1512, character path 1504, and character 1506. In optional embodiments, the in-game screen 1502 may include other items. The counter 1504 is at “10,” indicating there are 10 seconds left in the game. The status indicator 1506 prompts the user to “breathe in,” which will begin the game. The optimal breath path 1512 shows the breath that must be performed by the user. Once the user begins to breathe in, the marker 1510 will move along the optimal breath path 1502 to show the user if it is following along with the optimal breathing pattern. On the in-game display 1502 the character 1506 will move along the character path 1504, which is comprised of collectable points, in accordance with the user's breathing pattern, the object being to mimic the optimal breath path 1502 so that the character 1506 moves along the character path 1504 and collects the points along the way. Points accumulated are indicated in the score display 1508, with the user accumulating more points by breathing in accordance with the optimal breath path 1502 so that the character 1506 will move along the character path 1504 collecting points. The score display 1508 indicates that the user's current score is a “73.”

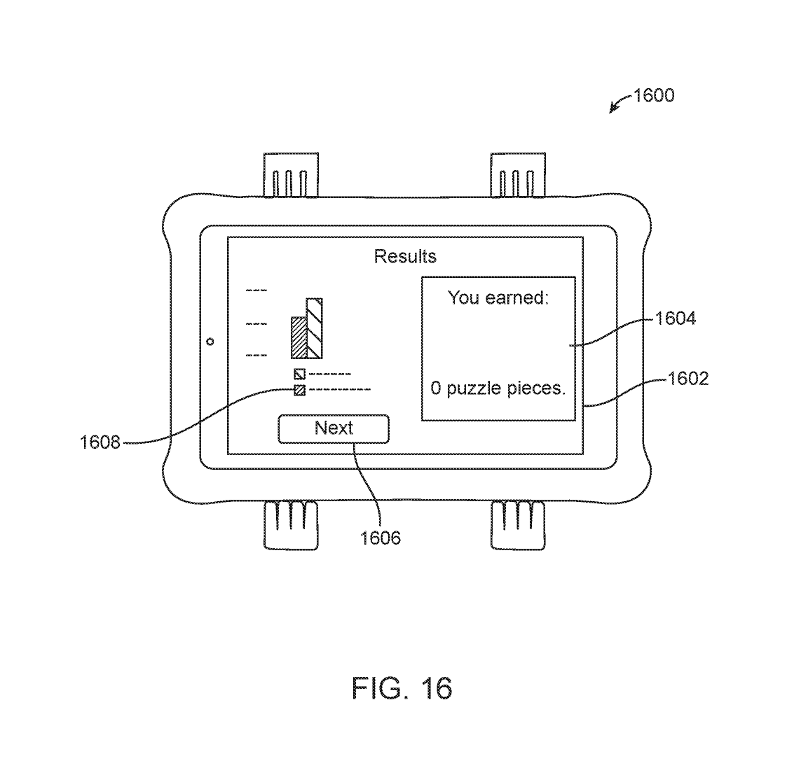

Referring now to FIG. 16, an exemplary results screen in accordance with one embodiment of the present invention is presented generally at 1600. The results screen 1602 comprises a point notification area 1604 that notifies the user of how many points it received in the particular game. In the current embodiment, the points are identified as puzzle pieces. However, in optional embodiments, the points may be in numeral format (e.g., 1289, 898) or may be in another form or measure that indicates a user's level of success for that particular game (e.g., money earned, fish caught). The result screen 1602 also comprises a pain chart 1608, which indicates the level of pain for the user in its prior game attempt as compared to the current game attempted. This allows the user to have a better understanding of its progress and may create an additional incentive for the user to perform better. The result screen 1602 comprises a next button 1606, which allows the user to exit the current game and return to the game selection game 1002, where it can select a new game or exit.

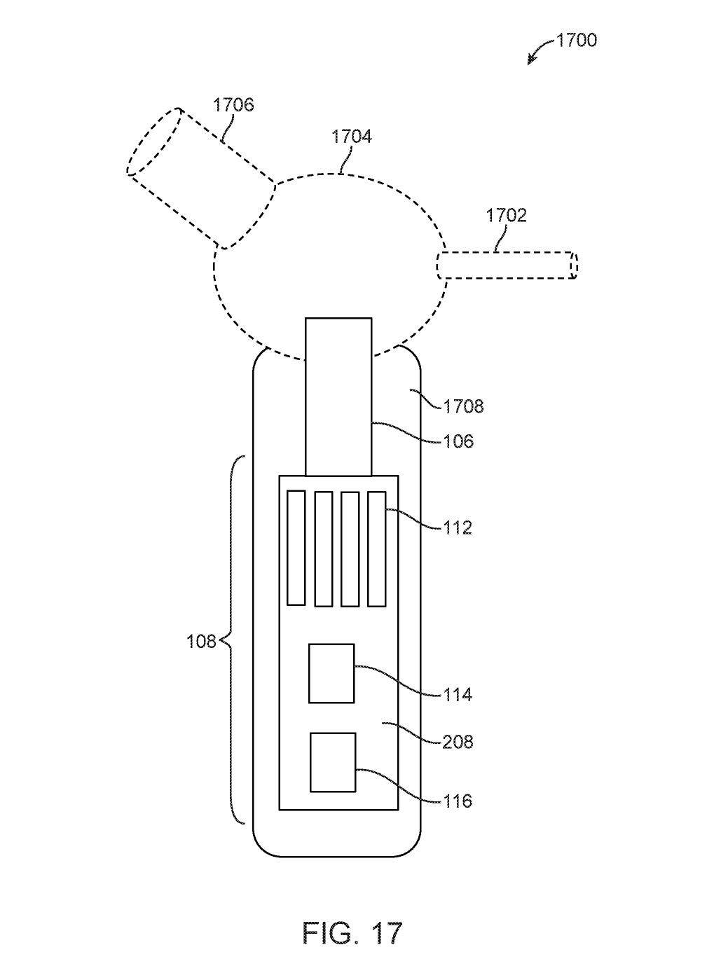

Referring now to FIG. 17, a diagram of the internal components of an optional embodiment of the respiratory therapy device in accordance with one embodiment of the present invention, in presented generally at 1700. In this optional embodiment, an incentive spirometer gaming controller is presented, which utilizes the internal hardware 108 and pressure sensor 106 of the respiratory therapy device 102. In the optional embodiment, the incentive spirometer gaming device 1700 contains a head 1704 and a body 1708. An outlet 1702 and mouthpiece 1706 are coupled to the head 1704. The body 1708 houses the internal hardware 108, which consist of a pressure sensor 106, random access memory 112, a central processing unit 114, and a communication interface 116, all of which is coupled to a circuit board 208. In the current embodiment, the communication interface 116 utilizes wireless Bluetooth® technology through which the incentive spirometer gaming device 1700 may communicate with other devices. In optional embodiments, the communication interface 116 may comprise wired technology or other forms of wireless technology, such as WiFi or Bluetooth®. The random-access memory 112 may store executable code as well as data that may be immediately accessible to the processor 114. The data stored in the random-access memory may include the games and performance scores, which were previously discussed in regard to FIGS. 12-14. The pressure sensor 106 is used to calculate the airflow and analyze a user's performance, thereby allowing for modifications to the user's games to create a more effective and efficient therapy session.

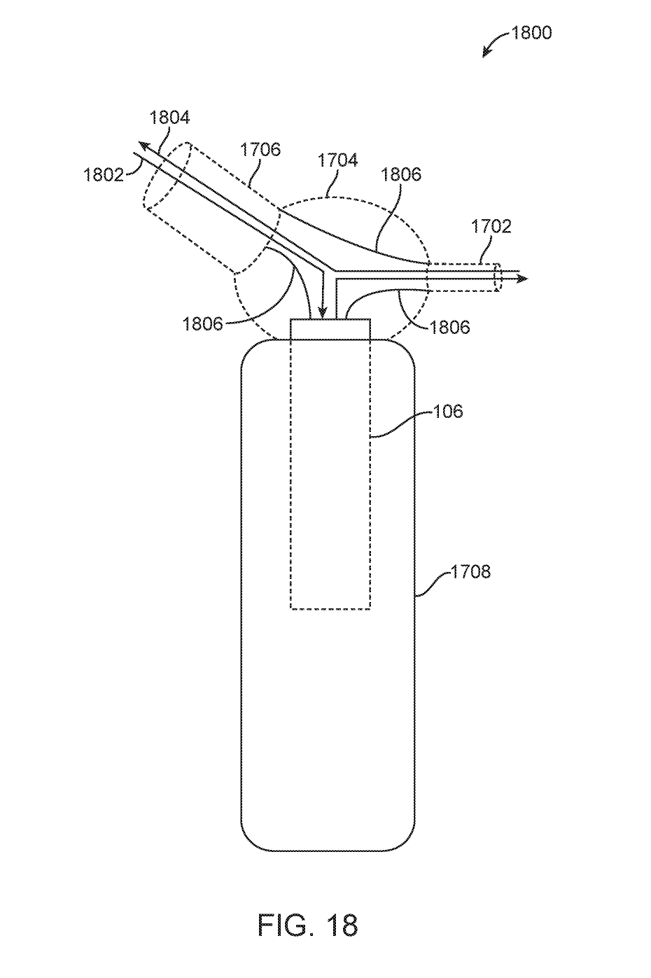

Referring now to FIG. 18, a flowchart illustrating the flow of air in and out of the incentive spirometer gaming device discussed in FIG. 17 in accordance with one embodiment of the present invention, is presented generally at 1800. The pressure sensor 106 is located within both the top 1704 and main body 1708. Depending on the in-game instructions provided on the smart device 118, the user will breathe air in and/or out of the incentive spirometer gaming device 1700 utilizing the mouthpiece 1706. When the user breathes air into the incentive spirometer gaming device 1700, meaning the user exhales into the incentive spirometer gaming device 1700, the air will travel through the mouthpiece 1706 and the top 1704 into the pressure sensor 106, where the pressure reading is calculated. Thereafter, the air then flows out of the top 1704 via the outlet 1702. The flow of air when users exhale into the mouthpiece 1706 is shown as 1802.

Still referring to FIG. 18, if the user is required to inhale air out of the incentive spectrometer gaming device 1700 via the mouthpiece 1706, meaning the user inhales air into the incentive spirometer gaming device 1700, the air will travel into the top 1704 via the outlet 1702 coupled to the top 1704, and the air will pass down into the pressure sensor 106 before being directed into the mouthpiece 1706 and into the user's mouth. The flow of air when a user inhales air from the mouthpiece 104 is shown as 1804. The flow of air within the top 1704 is guided through cavities 1806 within the top 1704. The cavities 1806 are coupled to the outlet 1702, mouthpiece 1706 and sensor 106 such that air traveling in and out of the top 1704 is guided directly to the sensor 106, mouthpiece 1706, and/or outlet 1702, as applicable, while reducing any air loss and therefore resulting in a more accurate reading with a more efficient and effective therapy session.

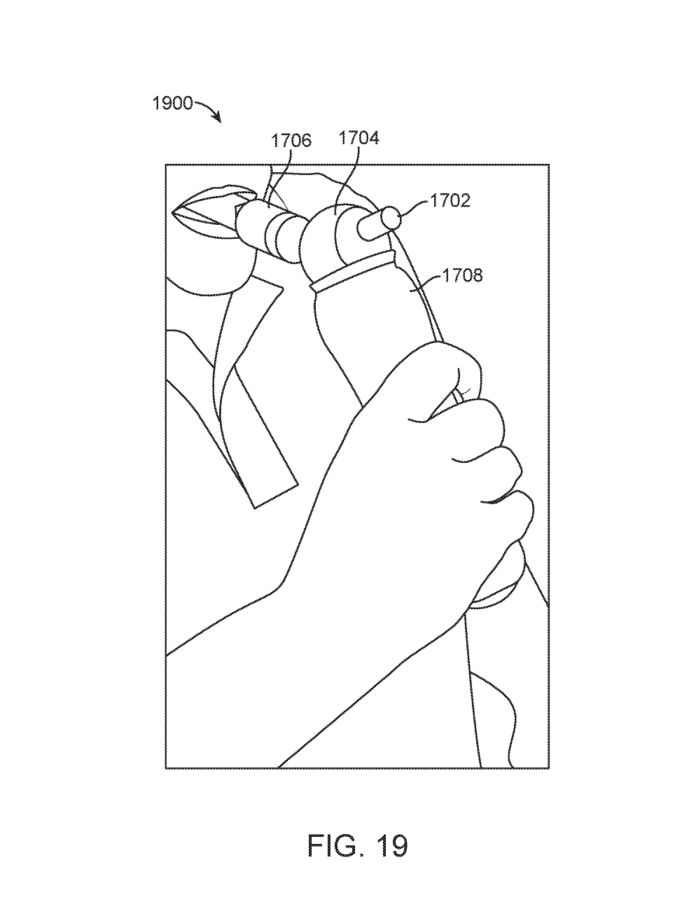

Referring now to FIG. 19, a side perspective view of the optional embodiment of the respiratory therapy device discussed in relation to FIG. 17 in accordance with one embodiment of the present invention, is presented generally at 1900. The incentive spirometer gaming device 1700 consists of a main body 1708 as well as a top 1704, with an outlet 1702 and mouthpiece 1706 coupled to the top 1704. The main body 1708 is designed with grooves shaped to conform to a human hand such that a user may grip the main body 1708 in the most natural position; this will in turn allow for a user to focus on the actual performance of the therapy games and less on its use of the incentive spirometer, thereby increasing the chance of having a more effective and efficient therapy session. The mouthpiece 1706 is used by the user to inhale and exhale air, which allows for playing of the respiratory therapy games such as those previously discussed in relation to FIG. 15. The air chamber 108 serves as a port through which air may flow in and out of the incentive spirometer 102. The flow of air and its relation to playing an incentive spirometry therapy game was previously discussed in relation to FIG. 15.

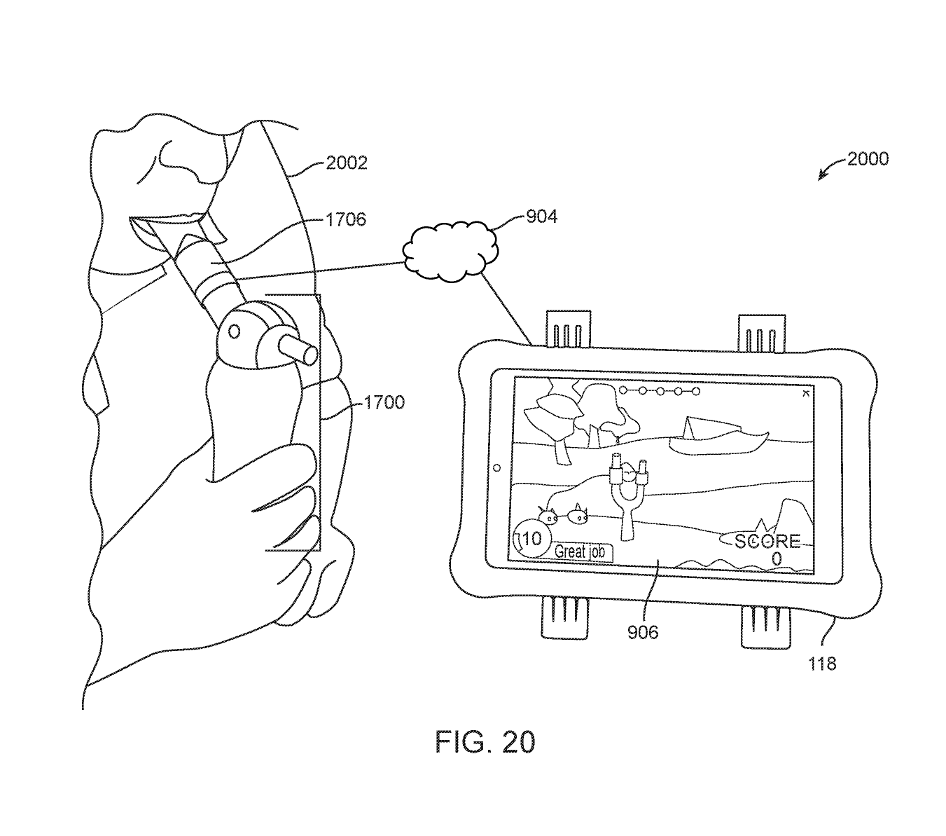

Referring now to FIG. 20, a perspective view diagram illustrating the use of the optional embodiment of the respiratory therapy device discussed in relation to FIG. 17 in accordance with one embodiment of the present invention, is presented generally. The user 2002 is depicted holding the incentive spirometer gaming device 1700 with their lips coupled to the mouthpiece 1706. The incentive spirometer 1700 is connected to the smart device 118 over a network 904 via a wireless Bluetooth® connection. The game the user is playing would be displayed on the screen 906. The specific game play was previously discussed in reference to FIGS. 12-14.

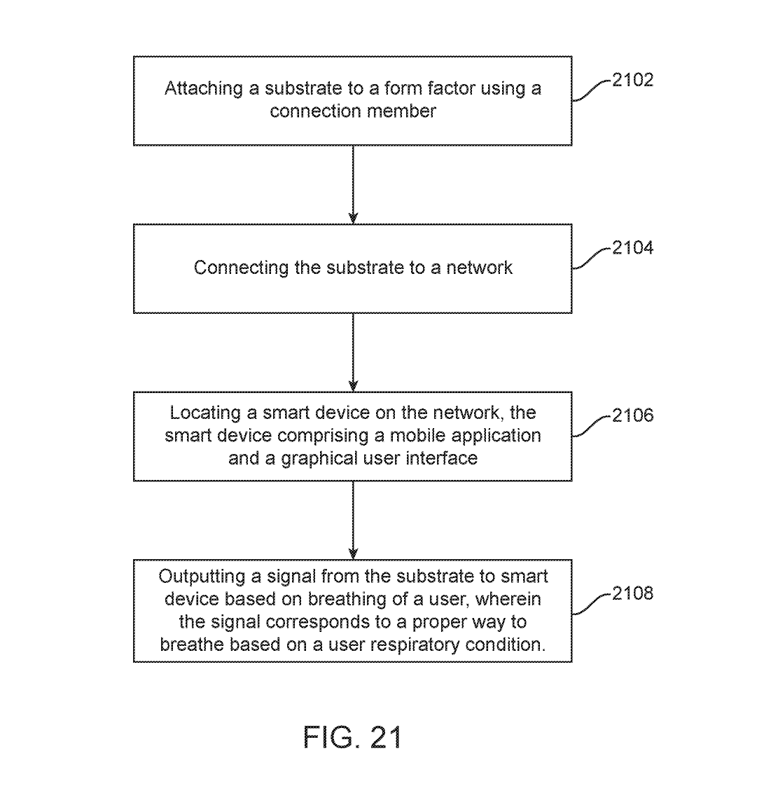

Referring now to FIG. 21, a step-wise diagram presenting a method for respiratory therapy that combines gaming and real-time feedback to guide users through proper respiratory techniques is presented generally at 2100.

At step 2102, a user attaches a substrate to a form factor using a gasket positioned proximate an outlet of the substrate, the gasket configured to form a seal with an opening of the form factor; the substrate comprising at least one sensor and at least one processor having wireless communications protocol.

At step 2104, a user connects the substrate to a network.

At step 2106, the device uses Bluetooth or other wireless protocol to locate a smart device on the network, the smart device comprising a mobile application and a graphical user interface.

At step 2108, a signal is output from the substrate to smart device based on breathing of a user, wherein the signal corresponds to a proper way to breathe based on a user respiratory condition.

Specific configurations and arrangements of the invention, discussed above regarding the accompanying drawing, are for illustrative purposes only. Other configurations and arrangements that are within the purview of a skilled artisan can be made, used, or sold without departing from the spirit and scope of the invention. For example, a reference to “an element” is a reference to one or more elements and includes equivalents thereof known to those skilled in the art. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures.

While the present invention has been described in connection with what are presently considered to be the most practical and preferred embodiments, it is to be understood that the present invention is not limited to these herein disclosed embodiments. Rather, the present invention is intended to mobile phone the various modifications and equivalent arrangements included within the spirit and scope of the appended claims.

Although specific features of various embodiments of the invention may be shown in some drawings and not in others, this is for convenience only. In accordance with the principles of the invention, the feature(s) of one drawing may be combined with any or all of the features in any of the other drawings. The words “including,” “comprising,” “having,” and “with” as used herein are to be interpreted broadly and comprehensively, and are not limited to any physical interconnection. Moreover, any embodiments disclosed herein are not to be interpreted as the only possible embodiments. Rather, modifications and other embodiments are intended to be included within the scope of the appended claims.

We claim:

1. A processor-based respiratory device for respiratory therapy that combines gaming and real-time feedback to guide a user through proper respiratory techniques, the device comprising:
a housing having a hollow interior, an outer wall, an inlet, and an outlet;
a radially inner portion defined by the housing to form a chamber, the chamber being positioned inside of the housing between the inlet and the outlet of the housing, the chamber being configured to allow air to flow from a user to a form factor when the user breathes into the chamber;
at least a sensor positioned within the chamber and electronically coupled to the processor, wherein the sensor is configured to measure airflow within the chamber;
a connection member positioned proximate the outlet, the connection member configured to form a seal with an opening of the form factor;
wherein the processor comprises a communications interface coupled to a network, the communications interface being configured to output a signal to a graphical user interface based on the airflow in the chamber.

2. The respiratory device of claim 1, wherein the connection member comprises a gasket positioned proximate the outlet, the gasket being configured to form a seal with an opening of the form factor, the gasket further comprising a gasket sensor configured to sense when the form factor is activated based on sensing the user shaking the form factor and further configured to connect to the smart device, which in activates a gaming menu.

3. The respiratory device of claim 1, wherein the at least a sensor is a pressure sensor is disposed at least partially in the chamber and the sensor is further coupled to the processor.

4. The respiratory device of claim 1, wherein the communications interface comprises a wireless connecting device configured to connect with the form factor, wherein the form factor is a smart device having a mobile application downloaded on it, wherein the mobile application comprises a plurality of respiratory training games, and the smart device is configured to receive signals from the communications interface whereby the user's breathing controls a game that is played on the smart device via the mobile application.

5. The respiratory device of claim 1, wherein the gasket is configured to mate the device with the plurality of form factors, and the gasket comprises an aperture formed of an elastic material configured to tightly seal the respiratory device to the breath opening of a form factor, wherein the gasket further comprises multiple tiers having different sized openings.

6. The respiratory device of claim 1, wherein the form factor comprises inhalers, spirometers, metered dosed inhalers nebulizers, and Positive Expiratory Pressure (PEP) devices.

7. The respiratory device of claim 1, further comprising a lower compartment configured to house the processor, communicates interface, and a plurality of electronic elements.

8. A system for respiratory therapy that combines gaming and real-time feedback to guide users through proper respiratory techniques, the system comprising:
a substrate comprising:
a housing having a hollow interior, an outer wall, an inlet and an outlet;
a radially inner portion defined by the housing to form a chamber, the chamber being positioned inside of the housing between the inlet and the outlet of the housing, the chamber being configured to allow air to flow from a user to a form factor when the user breathes into the chamber;
at least a sensor positioned within the chamber and electronically coupled to the processor, wherein the sensor is configured to measure airflow within the chamber and the processor is communicably coupled to a network;
a connection member positioned proximate the outlet, the connection member configured to form a seal with an opening of the form factor;
a smart device that is in communication with the processor of the substrate over the network, wherein the smart device comprises:
a graphical user interface; and
a smart device processor in communication with the network;
wherein the smart device comprises a mobile application configured to run software to allow the user to control a game that is played on the smart device using the substrate.

9. The system of claim 8, wherein the smart devices processor comprises a communications interface coupled to a network, and is configured to output the signal to a graphical user interface that is corresponds to the user's breathing into the substrate.

10. The system of claim 8, wherein the system comprises a gaming module that allows the user to select games that correspond to a respiratory condition, and wherein the games are configured to teach the user proper breathing techniques to alleviate an effect of the respiratory condition.

11. The system of claim 8, wherein the mobile application further comprising an assessment module configured to analyze the user's breathing techniques to determine a level of success with the game, wherein the assessment module is further configured use an input from the game play and configure an output based on the input to change the game for purposes of effective therapy.

12. The system of claim 8, wherein the mobile application further comprises a gaming module, the gaming module being configured to allow a user to select a game based on respiratory condition.

13. The system of claim 8, wherein the at least a sensor is a pressure sensor is disposed at least partially in the chamber and is further coupled to the processor, and the connection member comprises a gasket.

14. The system of claim 8, wherein the gasket is configured to mate the respirator device with the plurality of form factors, and comprises an aperture formed of an elastic material configured to tightly seal the respiratory device to the breath opening of the form factor, wherein the gasket further comprises multiple tiers having different sized openings, the gasket further comprising a gasket sensor configured to sense when the form factor is activated based on sensing the user shaking the form factor and further configured to connect to the smart device, which in activates a gaming menu.

15. The system of claim 8 wherein the form factor comprises inhalers, spirometers, and metered-dosed inhalers, nebulizers, Positive Expiratory Pressure (PEP) devices.

16. The system of claim 8, further comprising a lower compartment configured to house the processor, communicates interface, and a plurality of electronic elements.

17. A method for respiratory therapy that combines gaming and real-time feedback to guide users through proper respiratory techniques, the method comprising:
attaching a substrate to a form factor using a connection member positioned proximate an outlet of the substrate, the connection member configured to form a seal with an opening of the form factor; the substrate comprising at least one sensor and at least one processor having wireless communications protocol;
connecting the substrate to a network;
locating a smart device on the network, the smart device comprising a mobile application and a graphical user interface;
outputting a signal from the substrate to smart device based on breathing of a user, wherein the signal corresponds to a proper way to breathe based on a user respiratory condition.

18. The method of claim 17, further comprising allowing a user to select games that correspond to a respiratory condition, and wherein the games are configured to teach the user proper breathing techniques to alleviate an effect of the respiratory condition.

19. The method of claim 18, further comprising analyzing the user's breathing techniques to determine a level of success with the game and using an input from the game play and configure an output based on the input to change the game for purposes of effective therapy.

20. The method of claim 17, wherein the form factor comprise inhalers, spirometers, and metered-dosed inhalers nebulizers, Positive Expiratory Pressure (PEP) devices and the connection member comprises a gasket, the gasket further comprising a gasket sensor configured to sense when the form factor is activated based on sensing the user shaking the form factor and further configured to connect to the smart device, which in activates a gaming menu.
